SEQUENCE LISTING (2 sequences)

SEQ ID NO: 1 - protein, 1008 aa, Clostridium histolyticum
IANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYGYDIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKSTKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 2 - protein, 1008 aa, Clostridium histolyticum
IANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYGYDIQELVDKYQGAGILLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

(19) United States
(12) Patent Application Publication
(10) Pub. No.: US 2010/0159564 A1
(43) Pub. Date: Jun. 24, 2010

(54) Protease resistant recombinant bacterial collagenases

(76) Inventors: Francis E. Dwulet, Greenwood, IN (US); Andrew G. Breite, Fishers, IN (US); Robert C. McCarthy, Carmel, IN (US)

Correspondence Address: SANFORD J. PILTCH, ESQ., 1132 HAMILTON STREET, SUITE 201, ALLENTOWN, PA, 18101, US

(21) Appl. No.: 12/315,237

(22) Filed: Dec. 1, 2008

Related U.S. Application Data
(60) Provisional application No. 61/004,968, filed on Nov. 30, 2007.

(51) Int. Cl. C12N 9/52; C07H 21/04; C12N 5/10; C12N 5/071

(52) U.S. Cl. 435/220; 536/23.2; 435/325; 435/381

(57) ABSTRACT

The identification of the most sensitive sites of Clostridium histolyticum collagenase Class 1 to proteolysis by proteases present during the fermentation and purification of the enzyme is described. Culture supernatant obtained after fermentation of C. histolyticum is used as the starting material for further purification of the enzyme. Native collagenase Class 1 and its proteolytic fragments are partially purified by a combination of hydrophobic interaction and strong anion exchange chromatographies. The pools containing enriched levels of the proteolytic fragments are further purified by high performance anion exchange chromatography. These polypeptides are then characterized by Q-TOF mass spectroscopy. A total of three sensitive bonds are identified along with substitution and deletion strategies that will result in resistance of the enzyme to proteolytic degradation.

### FIELD OF THE INVENTION

This invention relates to recombinant collagenase enzymes which are resistant to cleavage by other proteases and their use in compositions for the enzymatic dissociation of biological tissues to recover viable cells from organs or tissue, wound debridement and tissue remodeling.

### BACKGROUND OF THE INVENTION

The enzymatic dissociation of organ or tissue into isolated cells or cell clusters is useful in a wide variety of laboratory, diagnostic and therapeutic applications. These applications involve the isolation of many types of cells for various uses, including recovery of microvascular endothelial cells for small diameter synthetic vascular graft seeding; hepatocytes for gene therapy, drug toxicology screening or extracorporeal liver assist devices; chondrocytes for cartilage regeneration; mesenchymal stem cells from adipose or other tissues for use in regenerative medicine; and islets of Langerhans for the treatment of insulin-dependent diabetes mellitus. Enzyme treatment works to fragment extracellular matrix proteins and other proteins that provide structural support to the tissue or organ. As collagen is the principle protein component in the tissue extracellular matrix, the enzyme collagenase in combination with other proteolytic enzymes (i.e., proteases) has been frequently used for tissue dissociation to recover viable single cells or cell clusters.

Collagenase has also been used for many years for wound debridement and more recently for non-surgical treatment of Depuytren's contracture, Peyronie's disease, and frozen shoulder syndrome, leading to remodeling of the tissue. In the former application, collagenase clears the wound, leading to faster wound repair and the minimization of scar formation. In the latter applications, collagenase breaks down collagen deposits, leading to improved anatomical function.

Different forms of bacterial collagenase derived from Clostridium histolyticum have been commercially available for a number of decades and are used to dissociate tissue leading to the release of single cells or cell clusters as well as for therapeutic applications. These “wild-type” collagenases are derived from cell culture supernatants recovered after fermentation of this organism. These supernatants are very heterogeneous containing a mixture of other proteases, primarily clostripain and a neutral protease, along with other secreted or released proteins from the cells. The function of wild-type collagenase for cell isolation, wound debridement and tissue remodeling is compromised by a number of factors including the variable concentration of enzymes, the concentration of endotoxins and the proteolytic degradation of the collagenase enzymes by proteases within the enzyme mixture and by endogenous proteases within or released from the tissue being dissociated or treated. Some of these issues have been addressed by the development of methods for the purification of the collagenase and blending it with other proteases. After a decade of use, these products are not manufactured with the consistency desired for research and/or therapeutic applications. What is needed is the identification of the current major causes of inconsistency and engineer enzymes or compositions that overcome these causes.

It is well known that C. histolyticum expresses two different collagenase enzymes, class 1 (C1) and class 2 (C2) that show different substrate specificity and gene sequences. Both gene sequences are expressed as single copies and are located in different portions of the genome. Several different molecular forms of both C1 and C2 ranging in mass from about 68 to 130 KDa are isolated or observed during purification steps as first reported by Van Wart and co-workers. Current evidence strongly suggests that these molecular forms are created by proteolysis of the native collagenase enzymes. The scientific literature is very unclear about the effects of proteolysis of C1 or C2 enzymes ability to degrade native collagen. Earlier literature indicated that there was no significant effect of proteolysis on the activity of the enzymes. However, the recent development of a more sensitive collagen degrading assay has identified that collagen degrading activity is significantly reduced after proteolysis. Variability in the extent of proteolytic damage to holoenzymes (i.e., intact enzyme including the zinc and calcium co-factors) during fermentation and purification has led to enzyme products with highly variable abilities to degrade collagen and thus perform effectively in tissue dissociation. The traditional approach to dealing with this problem is by selecting or verifying individual lots of collagenase after screening their function in tissue dissociation applications. Previous investigations have shown that each enzyme has three primary domain types as depicted in FIG. 1. With reference to FIG. 1, both C1 and C2 enzymes have a single relatively large catalytic domain responsible for cleaving native collagen. The C-terminal side of the catalytic domain is connected to a linking domain whose exact function is not yet understood. The C2 enzyme has two linking domains followed by a single collagen binding domain at the C-terminus. The C1 form, however, consists of a single linking domain followed by two collagen binding domains.

X-ray crystallographic information contributed by Matsushita and co-workers has shown that an isolated collagen binding domain has a very compact beta barrel three dimensional structure. A number of residues which are important for collagen binding have been identified and are all found on one surface of the domain. The remaining surface of the domain is almost entirely polar residues. This information coupled with preliminary x-ray data of Clostridial catalytic domains indicates that they also have a compact three dimensional structure. Thus it is probable that in solution the Clostridial collagenases would look like four balls on a string similar to the domain structure seen in immunoglobulins. With this type of structure it is probable that the spacing sequences connecting the domains may have little to no secondary structure. These loose random structures are often accessible to proteolytic enzymes. It is likely that at least some of the most sensitive cleavage sites should be found in these regions or other exposed loops in the domains.

HPLC and SDS-PAGE analyses on partially purified C. histolyticum fermentation supernatants indicate a number of breakdown products in a typical batch of raw material with some lots having very low levels of intact collagenase enzymes. Traditional chromatographic purification techniques have been unable to fully resolve many of the degraded forms from the intact forms without significant reduction in recovered enzyme. This means on the large production scale some of these breakdown products make their way into the final product. This could contribute to some of the lot-to-lot variability seen in tissue digestion reported by many end users. Preliminary analysis of these degradation patterns indicates that the bulk of the protease sensitive bonds in these two molecules are found in the collagenase C1 molecule.

In addition to the challenges in resolving the various molecular forms both manufacturers and users of collagenase have traditionally relied on the Wunsch assay or others using peptide substrates [e.g., FALGPA,] as a method of determining the activity of collagenase enzyme blends. These assays provide an incomplete assessment of the collagenase activity for two reasons. First, the assay is biased towards the C2 enzyme by a factor of near 50-fold over the C1 molecular form. For this reason, the quality of the C1 enzyme is not well characterized by this assay. Second, peptide substrates simply provide an assessment of the catalytic activity of the enzyme and not the ability to degrade native collagen. The ability of the enzyme to bind to collagen fibers through the collagen binding domain is crucial for the enzyme's ability to cleavage collagen and in turn initiate degradation of native collagen during tissue dissociation, wound debridement, or tissue remodeling procedures. If this feature of the enzyme composition is not characterized it provides an incomplete assessment as to the enzyme's ability to degrade native collagen.

Variable results are often obtained from applications using collagenase enzymes, leading some to believe that incompletely characterized an inconsistent product prevents commercialization of collagenase-based technology from reaching its full potential. There continues to be a need for a collagenase reagent that overcomes this problem.

### SUMMARY OF THE INVENTION

A solution for the problem described above is provided by creating modified (i.e. mutated) recombinant C1 collagenase molecules. These mutated enzymes contain amino acid substitutions, additions or deletions which remove or protect protease sensitive amino acids or sites and allow the collagenase enzymes to effectively resist proteolysis while retaining their biological activity. Proteolysis of C1 occurs during the fermentation and purification of natural or recombinant enzyme or in the application of these enzymes to recover cells from organs or tissue. It may also occur when these enzymes are used in wound debridement or as a therapeutic agent to remodel tissue.

The protease sensitive amino acid residues (sites) in C1 are determined by isolating the proteolyzed C1 forms and identifying the bonds of the collagenase which where degraded by bacterial proteases (e.g., clostripain and clostridial neutral protease). Degradation occurs during C. histolyticum culture and the purification process. The long fermentation time (24 to 48 hours) and elevated temperatures (≧30° C.) provide an opportunity for proteolysis to occur at the more sensitive sites. Clostripain is a sulfhydryl protease with a trypsin-like specificity for cleavage at the C-terminal side of arginine and to lesser extent lysine residues. Clostridial neutral protease is a family member of the bacterial metallo neutral proteases, which are zinc metallo proteases with specificity for cleavage at the amino terminal side of hydrophobic amino acids (preferably leucine and phenylalanine). These metallo neutral protease enzymes have specificity similar to chymotrypsin but cleave the bond at the amino terminal of the amino acid instead of at the carboxyl side. The identification of the sites sensitive to clostripain and clostridial neutral protease are of broad value because their specificity is similar to the majority of proteases secreted or released from bacterial and mammalian cells. Because this proteolysis is occurring on the native molecules, it is expected that residues located in ordered segments of secondary structure would be more resistant to proteolysis then the same residue in a spacing sequence with little secondary structure characteristics.

Once identified, there are several strategies that can be used to increase protease resistance. The first is to simply replace or delete the sensitive amino acid. A second strategy is to replace one or more amino acid residue around the sensitive residue. For trypsin sensitive residues placing an aspartic, glutamic or proline residue on the carboxyl terminal of the sensitive residue will also greatly reduce its sensitivity to proteolysis. Lastly, for complicated segments of sequence with potential multiple cleavage sites several residues may need to be replaced or deleted to confer protease resistance.

Engineering collagenases more resistant to proteolysis is accomplished by PCR site-directed mutagenesis methods to substitute, delete, or add DNA base pairs to the wild type C1 or C2 gene sequence, changing the amino acid sequence of the recombinant enzyme. A number of different amino acid residues, within an appropriate context, are used to replace the susceptible amino acid residues depending upon the nature of the amino acid residue. As an example, for trypsin sensitive amino acid residues, one could delete the susceptible amino acid (e.g., lys or arg), or replace the susceptible residues with a protease insensitive residue(s) (e.g., serine, threonine, glycine or other protease resistant residues). If suitable alternatives are not easily obtained, a region of the susceptible protein sequence can also be deleted or replaced with a random sequence.

The exact location of the most proteolytically sensitive residues is determined using a variety of analytical techniques including preparative column chromatographies for preliminary fractionation, analytical HPLC for fragment purification and Q-TOF MS analysis for sensitive mass determination. Also, enzyme activity analysis is used to understand the impact of proteolysis on enzyme function. Alterations of primary structure are expected to be kept to a minimum and will focus on the most sensitive sites to provide a more resistant enzyme, yet not alter the catalytic activity of the enzymes. A total of three major sites have been identified in the C1 molecule which appear to account for the bulk of the degradation of this enzyme.

### BRIEF DESCRIPTION OF THE DRAWINGS

For the purpose of illustrating the invention, there are shown in the drawings charts and graphs which are believed to be useful in understanding the invention, however, the invention is not limited to the precise arrangements and instrumentalities shown.

FIG. 1 is a graphic representation of the domain structure of Clostridium histolyticum collagenase C1 and C2 in which the numbers following the domain description indicate the number of amino acids in that particular domain.

FIG. 2 is a representative graphical trace of the strong anion exchange chromatographic separation of the preliminary purification of the C1 and C2 and their proteolytic fragments in which the numbers above peaks indicate retention time in minutes followed by percent integrated area of total.

FIG. 3A is a representative graphical trace of the deconvoluted Q-tof mass spectrum of the intact holo C1 enzyme.

FIG. 3B is a Coomassie stained SDS-PAGE gel of the holo C1 molecule.

FIG. 4A is a representative graphical trace of the deconvoluted Q-tof mass spectrum of the C1b enzyme.

FIG. 4B is a Coomassie stained SDS-PAGE gel of the C1b molecule.

FIG. 5A is a representative graphical trace of the deconvoluted Q-tof mass spectrum of the C1c enzyme.

FIG. 5B is a Coomassie stained SDS-PAGE gel of the C1c molecule.

FIG. 6A is a representative graphical trace of the deconvoluted Q-tof mass spectrum of the C1d enzyme.

FIG. 6B is a Coomassie stained SDS-PAGE gel of the C1d molecule.

FIG. 7A is a representation of the sequence alignments of the collagen binding domains from various clostridial species collagenases, N-terminal half of domain.

FIG. 7B is a representation of the sequence alignments of the collagen binding domains from various clostridial species collagenases, C-terminal half of domain.

FIG. 8A is a representation of the sequence alignments of the linking domains from various Clostridial species, N-terminal half of domain

FIG. 8B is a representation of the sequence alignments of the linking domains from various Clostridial species, C-terminal half of domain

### DETAILED DESCRIPTION OF THE PREFERRED EMBODIMENTS

The following detailed description is of the best presently contemplated mode of carrying out the invention. The description is not intended in a limiting sense, and is made solely for the purpose of illustrating the general principles of the invention. The various features and advantages of the present invention may be more readily understood with reference to the following detailed description taken in conjunction with the accompanying drawings.

### Analysis Tools

The collagen degrading assay (CDA) substrate is prepared by coupling FITC to soluble calf skin collagen fibrils using modifications of Baici's procedure as described previously above. The CDA assay is performed by adding 50 μL of 150 μg/mL FITC fibrils to wells containing no protein (blank) or collagenase in 100 μL of 100 mM Tris, 10 mM CaCl₂, pH 7.5. The 96 well solid black microplate is placed in a Bio-Tek FLx-800 fluorescent microplate reader and incubated at 35° C. for 60 minutes. Fluorescence readings are taken at 2.5 minute intervals using a 485 nm/528 nm filter set. Specific activities were calculated by dividing the enzyme units (fluorescent units per min) by the mg of protein per well determined by assuming 1 mg of purified C1 or C2 had an A₂₈₀=1.41. Analytical separation are performed by passing the collagenase sample over a 1 mL Mono-Q anion exchange column using a Beckman System Gold high pressure liquid chromatography (HPLC) instrument with a salt gradient. Each fraction is analyzed for A₂₈₀, CDA, and the molecular weight of the protein as determined by SDS-PAGE using 7.5% acrylamide gels. In each acrylamide gel, molecular weight markers are run at 15, 35, 50, 75, 100, and 150 kDa. The relative percentage of protein found in the bands in each lane and their apparent molecular weight are determined by using a Kodak Image Station 440 CF equipped with ID1 software. Pools and fractions were additionally characterized using the Wunsch peptide as a substrate. This is a well characterized protocol and a detailed description of the protocol can be found in U.S. Pat. Nos. 5,753,485, 5,830,741, 5,952,215 and 5,989,888 (Dwulet, et al.) which description is incorporated herein by reference.

### Determining Sensitive Amino Acid Residues

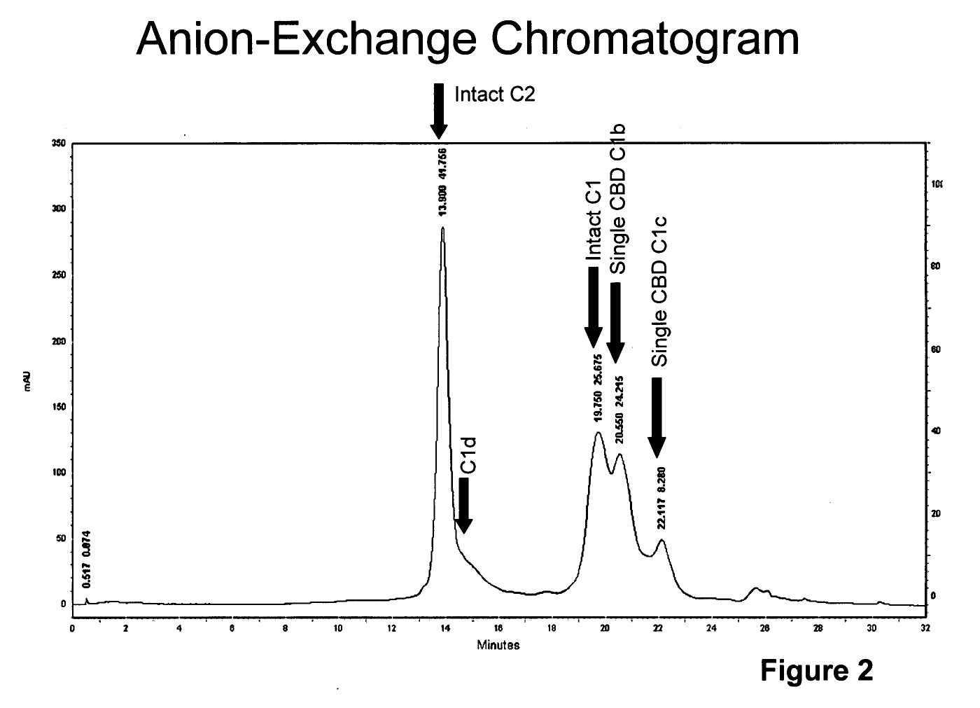

Natural C1 and C2 along with proteolyzed forms are purified using minor modifications of several types of chromatography resin chemistries previously used in the patent and scientific literature with the final step being passage over an anion exchange resin with a representative chromatogram as shown in FIG. 2. On an analytical Mono Q HPLC column C2 elutes near 13.9 minutes, while the intact C1 eluted near 20 minutes. Depending on the sample, two additional peaks on the back side of the C1 peak are observed, but not entirely resolved. These molecular forms, designated C1b and C1c elute approximately one and two minutes after the intact C1 peak, respectively. The C1d peak is found on the back side of the C2 peak and contains two major components of approximately 78 and 88 kDa as determined by Q-TOF mass spectrophotometry analysis.

The three pools containing the proteolyzed forms of the C1 enzyme were then further purified using analytical ion exchange chromatography. Analysis of the isolated fractions by a variety of techniques indicated the C1 protein contains at least four distinct populations including the intact 113 kDa C1 containing two collagen binding domains, and two forms of degraded enzyme with only 1 collagen binding domain. Lastly a C1 form was recovered and identified as having no collagen binding domains. Wilson's x-ray crystallographic data on the carboxyl terminal collagen binding domains of C1 indicate that this domain is a functionally independent structural unit. Our observations and all literature on these proteolyzed forms of C1 are consistent with the conclusion that when then the peptide bond between the two collagen binding domains is cleaved, the terminal collagen binding domain dissociates from the remainder of the enzyme. This apparent lack of structure between the two collagen binding domains allows for great flexibility in structure modification to include substitutions, deletions and extensions.

After the final purification on the preparative scale anion exchange column selected fractions are further purified by analytical Mono Q anion exchange column chromatography using a very shallow gradient. Those fractions containing primarily one component are dialyzed extensively against water and 1 mM EDTA to remove Zn²⁺ and Ca²⁺ ions that could hamper the ionization step in the mass spectrometry analysis. The dialyzed samples are then concentrated and stored frozen prior to further analysis.

### Example 1

### Purification of Collagenase C1 and C2 and their Proteolytic Fragments from Natural Fermentation Media

The crude collagenase in this work is prepared using minor modifications of the protocol of Warren & Gray. Analysis of this material and crude collagenase samples from different vendors by analytical Mono Q HPLC revealed the same approximate distribution of holo and proteolyzed collagenase enzymes. This material differs from the other crude collagenase samples in having a lower than average clostripain and neutral preotase concentrations classifying it as a low protease crude collagenase material.

Preliminary enzyme purification was accomplished by hydrophobic interaction chromatography on a hexyl agarose support similar to the protocol reported in the U.S. Patent Application Publication US 2007/0224183 A1 by Sebatino, et al. Both sodium chloride and ammonium sulfate were found to be able to induce binding of the collagenase while allowing the bulk of the fermentation by-products and clostripain to pass through the column unbound. Depending upon the vendor and type of resin used, at around neutral pH, a sodium chloride concentration of about 4.0 M or an ammonium sulfate concentration of about 1.0 M were both effective of binding the collagenase enzymes to the this support. For this work a bis-Tris buffer is used but other buffer salts are expected to work but have not been characterized. In this work there is little effect of pH on the binding of the collagenase to this support. However, other supports and starting materials may require modification to optimize the recoveries and purification factors. The collagenase enzymes were eluted with the same buffer but with no added salt.

The sample is then concentrated and desalted by dialysis or buffer exchange using a stirred cell or tangential flow filtration unit. The collagenase sample is exchanged into a low salt buffer at neutral to slightly alkaline pH. Both Tris and Glycylglycine buffers have both been found effective but other buffers are expected to work but have not been evaluated. Both Q Sepharose Fast Flow and Q Sepharose High Performance (GE Healthcare) have been found to be effective and other resins with similar properties are expected to work as well. Preliminary separation was accomplished using a gradient elution with sodium chloride similar to the protocol reported in the Sebatino, et al. Publication. A representative anion-exchange chromatogram is depicted in FIG. 2. Across the entire chromatogram fractions were collected and analyzed by SDS-PAGE and collagenase assays.

### Example 2

### Analysis of Natural C. Histolyticum Collagenase C1 Mass Spectra Data

The two gene derived amino acid sequences of the mature C. histolyticum C1 enzyme are shown in TABLE 1 appearing below. The complete sequence of Matsushita is shown in its entirety. The sequence reported by Burtscher contains only four differences and are identified at the appropriate positions. Both sequences code for mature proteins of 1008 amino acid residues with an empirical mass difference of 2 daltons (atomic mass units AMUs).

TABLE 1
C1 AMINO ACID PROTEIN SEQUENCE

| | | | | | |
|---|---|---|---|---|---|
| 10 | 20 | 30 | 40 | 50 | 60 |
| IANTNSEKYD | FEYLNGLSYT | ELTNLIKNIK | WNQINGLFNY | STGSQKFFGD | KNRVQAIINA |
| 70 | 80 | 90 | 100 | 110 | 120 |
| LQESGRTYTA | NDMKGIETFT | EVLRAGFYLG | YYNDGLSYLN | DRNFQDKCIP | AMIAIQKNPN |
| 130 | 140 | 150 | 160 | 170 | 180 |
| FKLGTAVQDE | VITSLGKLIG | NASANAEVVN | NCVPVLKQFR | ENLNQYAPDY | VKGTAVNELI |
| 190 | 200 | 210 | 220 | 230 | 240 |
| KGIEFDFSGA | AYEKDVKTMP | WYGKIDPFIN | ELKALGLYGN | ITSATEWASD | VGIYYLSKFG |
| 250 | 260 | 270 | 280 | 290 | 300 |
| LYSTNRNDIV | QSLEKAVDMY | KYGKIAFVAM | ERITWDYDGI | GSNGKKVDHD | KFLDDAEKHY |
| 310 | 320 | 330 | 340 | 350 | 360 |
| LPKTYTFDNG | TFIIRAGDKV | SEEKIKRLYW | ASREVKSQFH | RVVGNDKALE | VGNADDVLTM |
| 370 | 380 | 390 | 400 | 410 | 420 |
| KIFNSPEEYK | FNTNINGVST | DNGGLYIEPR | GTFYTYERTP | QQSIFSLEEL | FRHEYTHYLQ |
| 430 | 440 | 450 | 460 | 470 | 480 |
| ARYLVDGLWG | QGPFYEKNRL | TWFDEGTAEF | FAGSTRTSGV | LPRKSILGYL | AKDKVDHRYS |
| 490 | 500 | 510 | 520 | 530 | 540 |
| LKKTLNSGYD | DSDWMFYNYG | FAVAHYLYEK | DMPTFIKMNK | AILNTDVKSY | DEIIKKLSDD |
| 550 | 560 | 570 | 580 | 590 | 600 |
| ANKNTEYGYD | IQELADKYQG | AGIPLVSDDY | LKDHGYKKAS | EVYSEISKAA | SLTNTSVTAE |
| | V | L | | | |
| 610 | 620 | 630 | 640 | 650 | 660 |
| KSQYFNTFTL | RGTYTGETSK | GEFKDWDEMS | KKLDGTLESL | AKNSWSGYKT | LTAYFTNYRV |
| 670 | 680 | 690 | 700 | 710 | 720 |
| TSDNKVQYDV | VFHGVLTDNA | DISNNKAPIA | KVTGPSTGAV | GRNIEFSGKD | SKDEDGKIVS |
| | G | | | | |
| 730 | 740 | 750 | 760 | 770 | 780 |
| YDWDFGDGAT | SRGKNSVHAY | KKAGTYNVTL | KVTDDKGATA | TESFTIEIKN | EDTTTPITKE |
| 790 | 800 | 810 | 820 | 830 | 840 |
| MEPNDDIKEA | NGPIVEGVTV | KGDLNGSDDA | DTFYFDVKED | GDVTIELPYS | GSSNFTWLVY |
| 850 | 860 | 870 | 880 | 890 | 900 |
| KEGDDQNHIA | SGIDKNNSKV | GTFKSTKGRH | YVFIYKHDSA | SNISYSLNIK | GLGNEKLKEK |
| | | A | | | |
| 910 | 920 | 930 | 940 | 950 | 960 |
| ENNDSSDKAT | VIPNFNTTMQ | GSLLGDDSRD | YYSFEVKEEG | EVNIELDKKD | EFGVTWTLHP |
| 970 | 980 | 990 | 1000 | 1008 | |
| ESNINDRITY | GQVDGNKVSN | KVKLRPGKYY | LLVYKYSGSG | NYELRVNK | |

In a bacterial protein of this size it is not surprising that polymorphisms are seen especially since the respective works were accomplished on opposite sides of the world using two different strains of the bacteria.

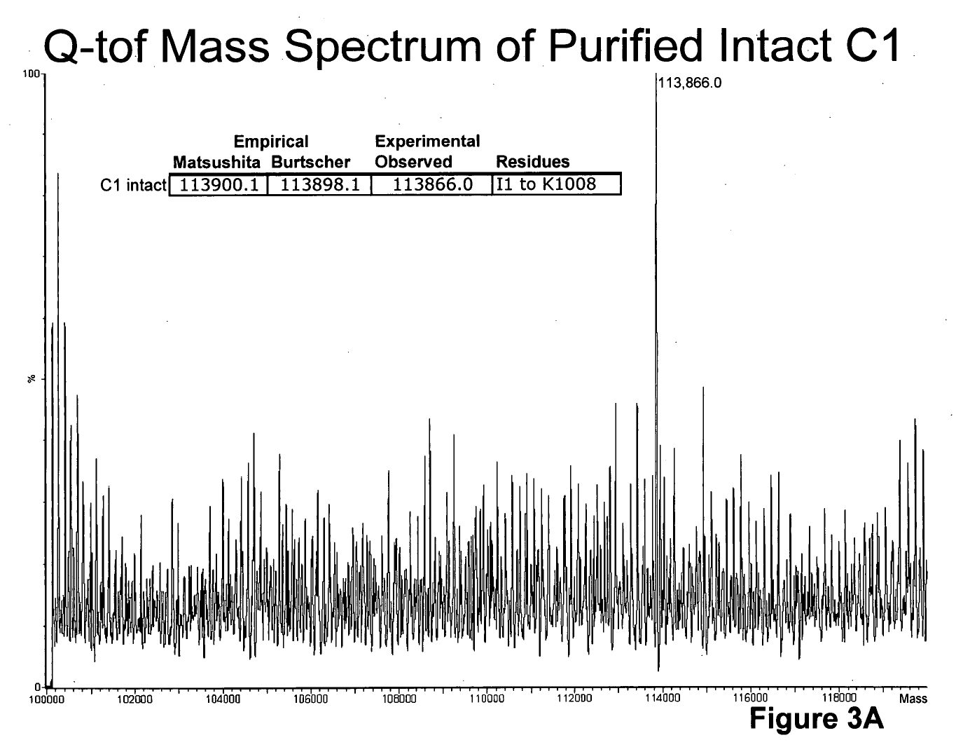

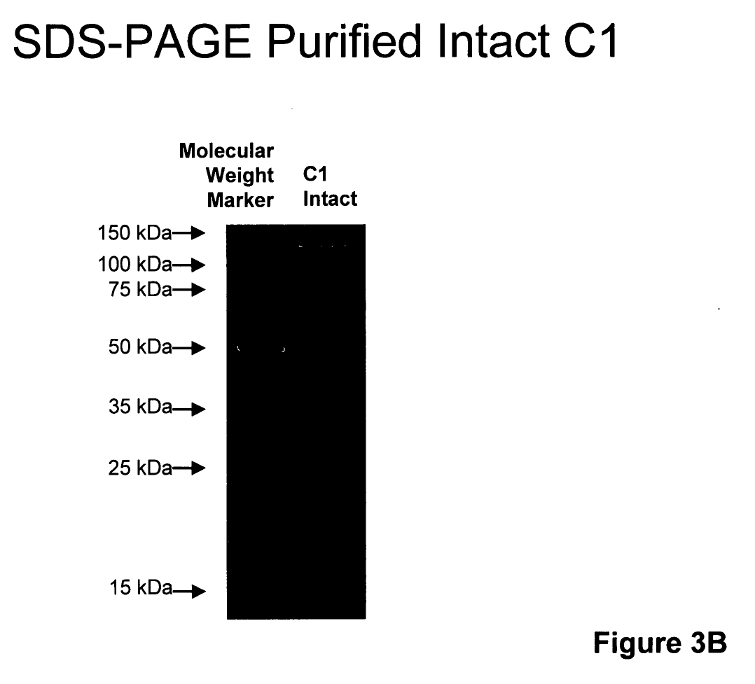

The deconvoluted mass spectra results observed here along with a representative SDS-PAGE gel can are shown in FIGS. 3A, 3B. The observed parental molecular mass of our isolated protein is 113,866 daltons [FIG. 3B], which is 34 and 32 AMUs lighter than the calculated masses of the Matsushita and Burtscher sequences, respectively. If this mass difference represents a real molecular difference then the most likely explanation for this difference is that the strain used in this work contains one or more polymorphisms which are different from the two reported sequences. A number of replacements are possible and two of several examples are that two conserved serine residues have been replaced by alanine residues or a conserved threonine residue is replaced by an alanine residue. A number of other replacements are possible and the exact modifications are unimportant because they are expected to have a minimal impact on the determination of the fragmentation sites of the molecule.

### Example 3

### Analysis of Natural C. histolyticum Collagenase Class 1b Mass Spectra Data

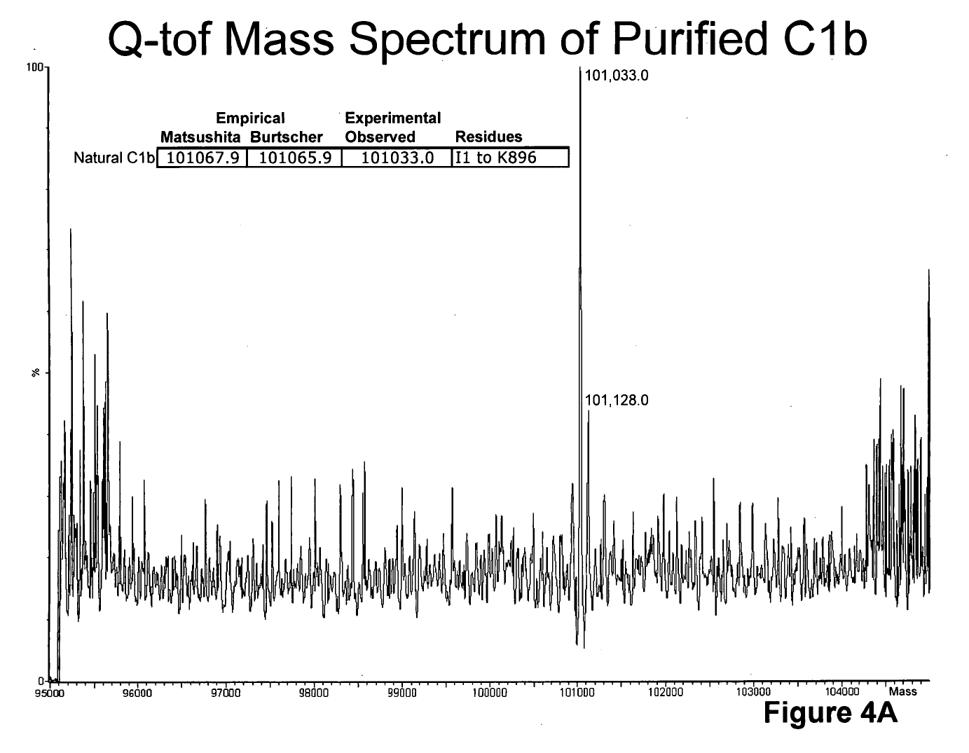

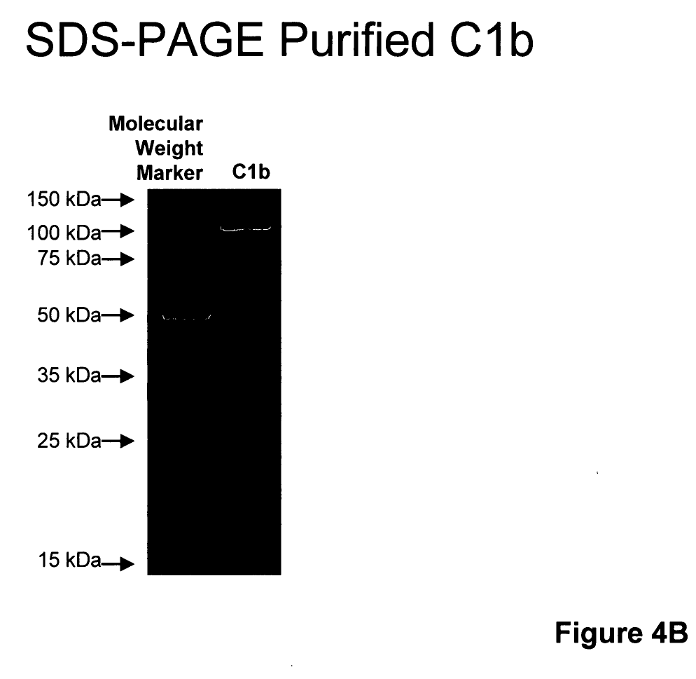

After purification a highly homogeneous sample of the C1b protein was recovered. The deconvoluted mass spectra results observed here along with a representative SDS-PAGE gel is shown in FIGS. 4A, 4B. The observed mass of this enzyme fragment is 101,033 AMUs is 12,833 AMUs less than the parent molecule. Fragmentation of the Matsushita and Burtscher proteins between lysine 896 and leucine 897 would provide fragments with molecular masses of 101,066 and 101,064 AMUs, which represent losses of 12,834 AMUs, respectively. Within the error of analysis this is considered a very high probability match. From x-ray crystallographic analysis this proteolysis site is located between the two collagen binding domains of the molecule. Collagen degrading activity analysis of this molecule is consistent with the observations of Matsushita that showed the loss of the second collagen binding domain results in a significant reduction in the ability of the molecule to bind to collagen.

On a practical level, the extreme difficulty in resolving this proteolytic form from the holoenzyme by standard chromatographic techniques appears to be a significant contributor to enzyme variability. Because of the nature of the bond being proteolyzed (lys-leu) and the enzymes involved (clostripain and Clostridial neutral protease) it is impossible to tell at this time which enzyme is responsible because either enzyme could proteolyze this bond. To protect this region several options are available. The first is to replace both residues and the second is to delete both residues. A third approach is to replace the leucine with at a minimum one asp, glu or pro residue. Neither of these three residues can be proteolyzed by Clostridial neutral protease and they significantly reduce the sensitivity of the preceding lysine residue to trypsin like cleavage.

### Example 4

### Analysis of Natural C. Histolyticum Collagenase Class 1c Mass Spectra Data

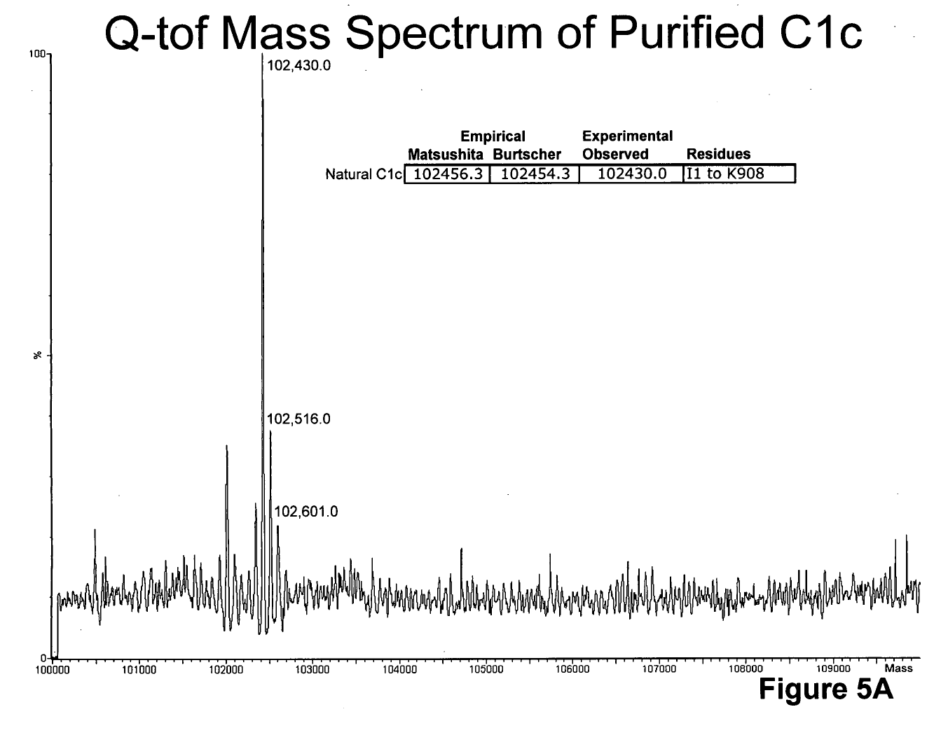

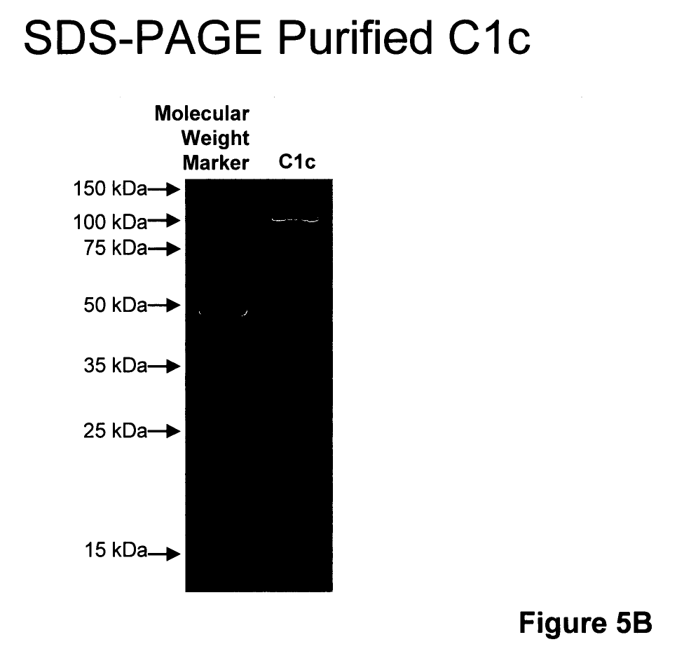

After purification a highly homogeneous sample of the C1c protein was recovered. The deconvoluted mass spectra results observed here along with a representative SDS-PAGE gel is shown in FIGS. 5A, 5B. The observed mass of this enzyme fragment is 102,430 AMUs is 11,436 AMUs less than the parent molecule. Fragmentation of the Matsushita and Burtscher proteins between lysine 908 and alanine 909 would provide fragments with molecular masses of 102,456 and 102,454 AMUs, which represent losses of 11,444 AMUs, respectively. Again, within the error of analysis this is considered a very high probability match. From x-ray crystallographic analysis this proteolysis site is located in an unstructured segment near the amino terminal of the second collagen binding domain of the molecule. Collagen degrading activity analysis of this molecule is consistent with the observations of Matsushita that showed the loss of the second collagen binding domain results in a significant reduction in the ability of the molecule to bind to collagen.

On a practical level this proteolytic form is somewhat easier to remove from the holo enzyme but requires high resolution resins which are expensive, slow and have reduced capacity as compared to the standard chromatographic resins and appears to be an additional contributor to product variability. Because of the nature of the bond being proteolyzed (lys-ala) and the enzymes involved (clostripain and Clostridial neutral protease), it is impossible to tell with certainty which enzyme is responsible because either enzyme could proteolyze this bond.

Cleavage at alaninine residues by thermolysin like enzymes occurs infrequently, however, this enzyme has been also classified as an elastase. Elastases are known to have an enhanced affinity for cleavage at alanine residues and so either enzyme could be responsible for this fragmentation. To protect this region several options are available which are identical to the approaches used for the C1b cleavage site. The first is to replace both residues and the second is to delete both residues. A third approach is to replace the alanine with at a minimum one asp, glu or pro residue. Neither of these three residues can be proteolyzed by Clostridial neutral protease and they significantly reduce the sensitivity of the preceding lysine residue to trypsin like cleavage.

### Example 5

### Analysis of natural C. histolyticum Collagenase Class 1d Mass Spectra Data

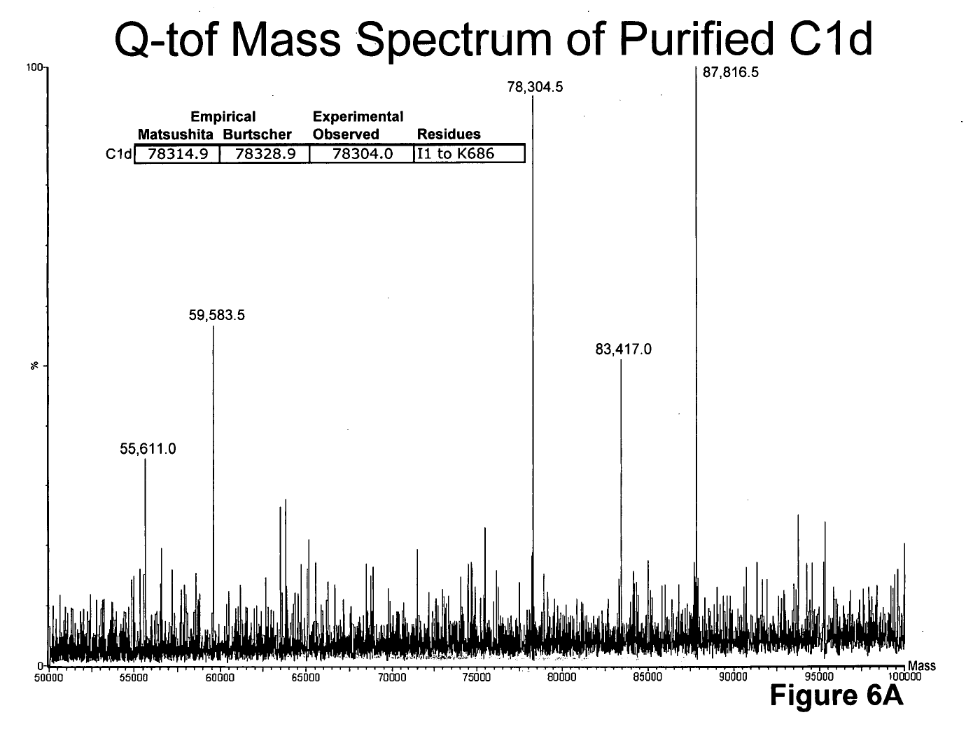

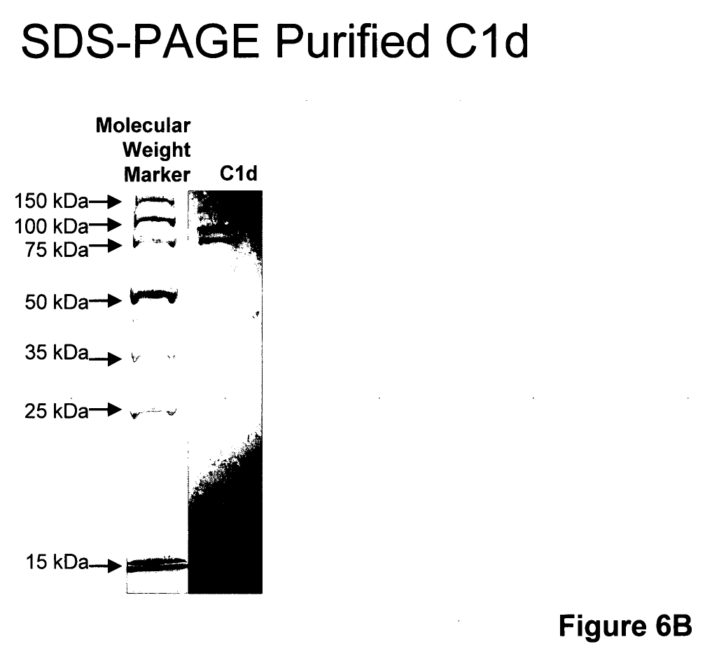

This fragment is recovered between the holo C1 and C2 pools eluted from the strong anion exchange chromatography column. After re-purification an enriched sample of the C1d protein 78 kDa form was partially separated from the 88 kDa form. The 78 kDa form was identified as having a FALGPA activity consistent with a C1 collagenase catalytic unit and collagen degradation activity analysis indicates little to no collagen degrading activity. The deconvoluted mass spectra results observed here along with a representative SDS-PAGE gel can be seen in FIGS. 6A, 6B. The observed mass of this enzyme fragment is 78,304 AMUs is 35,562 AMUs less than the parent molecule. Because of the activity against low molecular weight peptide substrates and the poor activity against native collagen it is probable that this fragment is derived from the catalytic domain of the C1 enzyme. Fragmentation of the Matsushita and Burtscher C1 proteins between lysine 686 and alanine 687 would provide fragments with molecular masses of 78,314 and 78,328 AMUs which represent losses of 35,586 and 35,570 AMUs, respectively. The mass difference between our peptide and the calculated sequences is within the error of analysis and is considered a high probability match. From preliminary structure analysis this proteolysis site is located at what appears to be a spacing segment between the catalytic domain and the linking domain.

On a practical level this proteolytic form is somewhat easier to remove from the holo C2 enzyme but requires high resolution resins which are expensive, slow and have reduced capacity as compared to the standard chromatographic resins, and further, appears to be an additional contributor to product variability. Because of the nature of the bond being proteolyzed (lys-ala) and the enzymes involved (clostripain and Clostridial neutral protease) it is impossible to tell at this time which enzyme is responsible because either enzyme could proteolyze this bond for the same reasons noted for the C1c peptide. To protect this region several options are available which are identical to the approaches used for the C1b cleavage site. The first is to replace both residues and the second is to delete both residues. A third approach is to replace the alanine with at a minimum one asp, glu or pro residue. Neither of these three residues can be proteolyzed by Clostridial neutral protease and they significantly reduce the sensitivity of the preceding lysine residue to trypsin like cleavage.

### Example 6

### Homologies of Clostridium Collagen Binding Domains

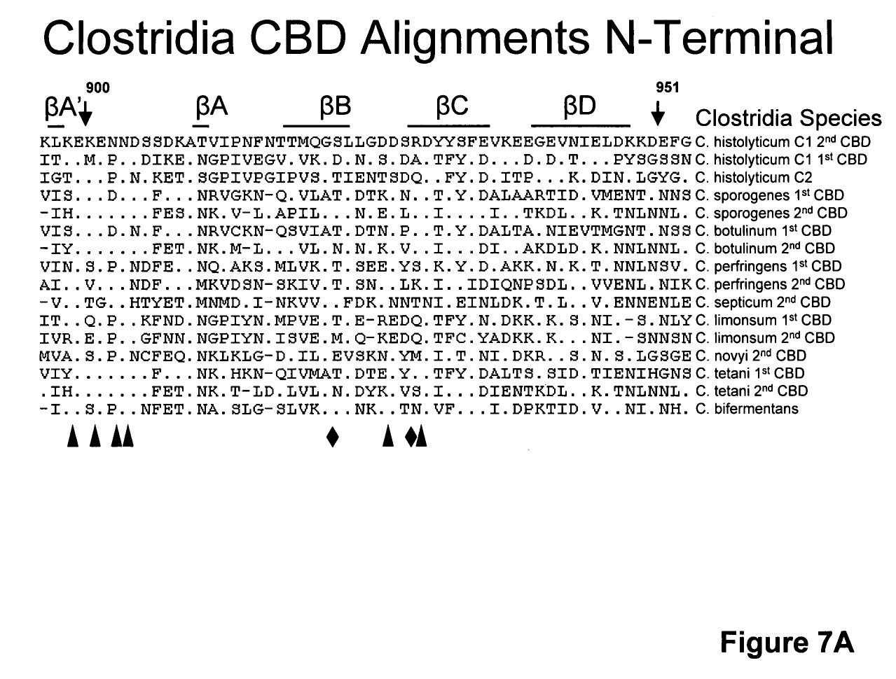

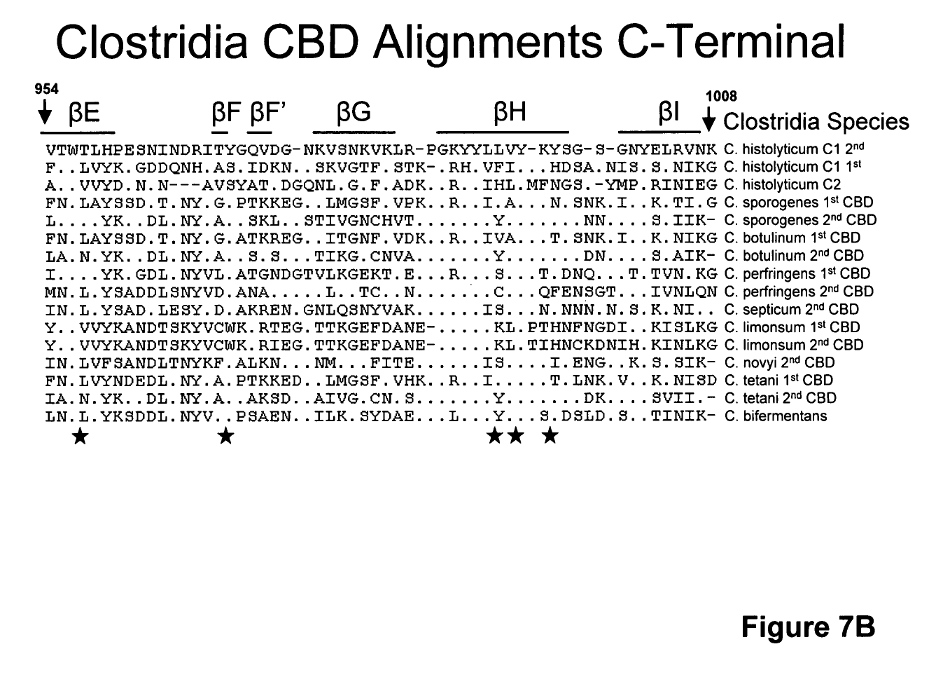

The gene derived amino acid protein sequences of a number of collagenase Class 1 proteases have been determined from a number of Clostridial species and the alignment of their collagen binding domains can be found in FIGS. 7A, 7B. In order to properly interpret the chart of FIG. 7A, the NUMBERS at the top of the chart refer to an amino acid residue number in the full length protein sequence of the Class C1 C. histolyticum collagenase, the DASHES refer to an amino acid deletion, the DOTS indicate an identical amino acid to the residue in the first sequence appearing in the first line of the chart, the LINES above the sequence refer to secondary amino acid structure as determined by Matsushita, the TRIANGLES refer to amino acid side chains involved in calcium bonding, and the diamonds refer to amino acid carbonyl carbons involved in calcium bonding. For the chart of FIG. 7B, the NUMBERS at the top of the chart refer, again, to an amino acid residue number in the full length protein sequence of the Class C1 C. histolyticum collagenase, the DASHES refer to an amino acid deletion, the DOTS indicate an identical amino acid to the residue in the first sequence appearing in the first line of the chart, the LINES above the sequence refer to secondary amino acid structure as determined by Matsushita, the STARS indicate residues critical for collagen binding as determined by Matsushita.

These sequences have been aligned using BioEdit and ClustalW alignment algorithms to maximize homology while minimizing insertions and deletions. Observing the two cleavage sites determined in the C. histolyticum second collagen binding domain, two very different patterns of homology are seen. The Lys-Leu sequence (position 896-897) is only seen in the second collagen binding domain of the Clostridial C1 gene. The only other lysine residue seen in this position is in the C. tetani second collagen binding domain and it is followed by an isoleucine residue that is known to significantly reduce proteolysis rates of that bond through the personal experience of the inventors. All other sequences have either deleted the lysine or replaced it with a hydrophobic amino acid residue (ala, val, ile or met). C. histolyticum is the only strain to have a leucine at position 897, with ile and val being the predominant residues. It would seem that multiple substitutions are possible at this location and that the selection of substitution or deletion will be determined by susceptibility to other proteases.

The second proteolytically sensitive sequence in this region is the Lys-Ala sequence (position 908-909). In this region every Clostridia C1 collagen binding domain has an alanine residue at the position analogous to position 909 while half of the collagen binding domains have a lysinine or arginine residue at the position analogous to position 908. The other domains have either an asp, asn, glu, gln, ser, or thr residue. Because these two sensitive bonds flank the amino and carboxyl sides of a calcium binding site responsible for protein stabilization the impact of substitutions in this region must be characterized carefully.

### Example 7

### Homologies of the Linking Region Between the Catalytic Domain and the First Collagen Binding Domain of the C1 Molecule

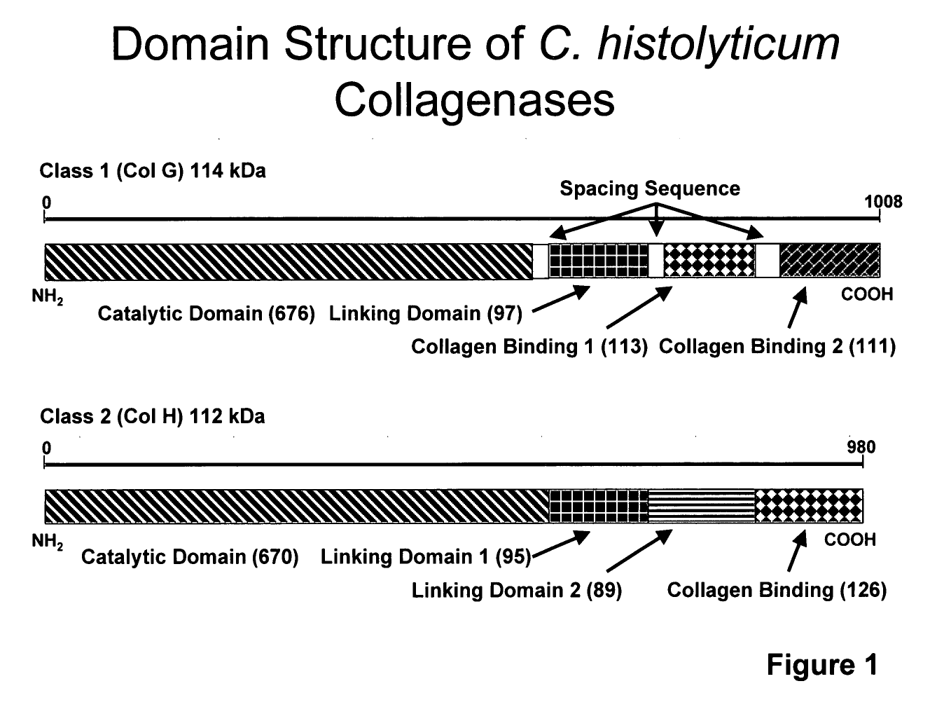

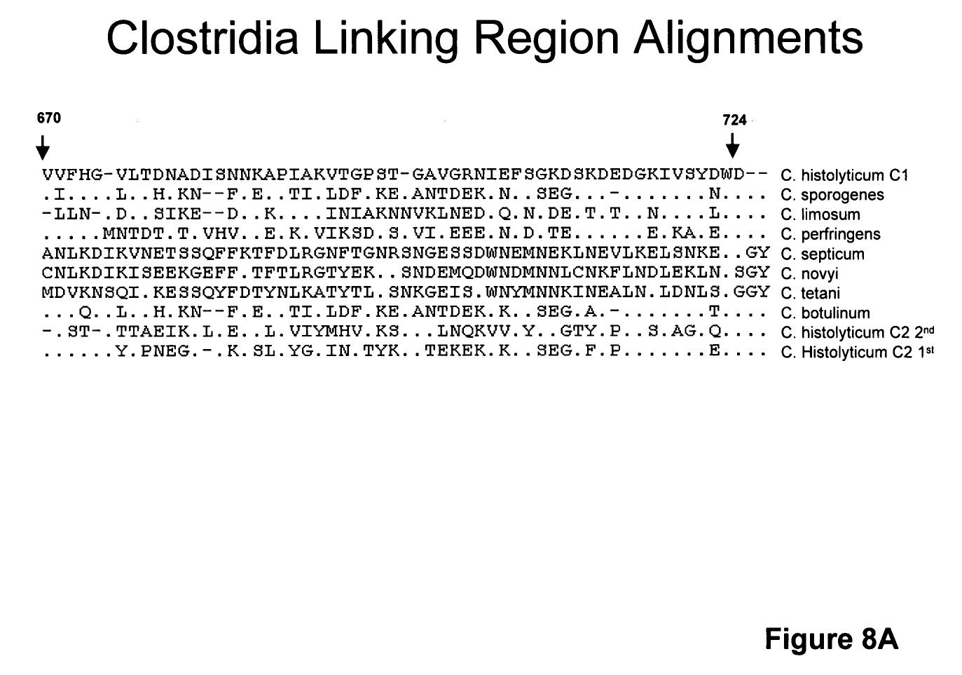

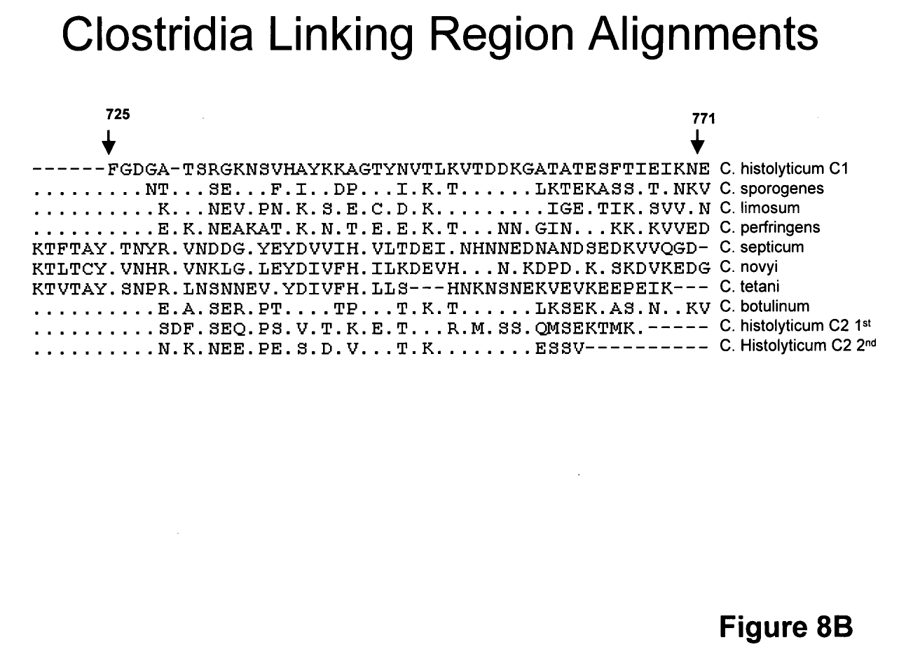

Within the Clostridial C1 enzyme located between the catalytic domain and the first collagen binding domains there exists a region of amino acid sequence identified as the linking domain [See, FIG. 1.]. It is about 100 amino acids long (about the same size as the collagen binding domains), but as of yet has no identified function. All Clostridial collagenase enzymes have at least one of these regions. Alignment of these regions can be seen in FIGS. 8A, 8B. In order to properly interpret the chart of FIG. 8A, the NUMBERS refer to an amino acid residue number of the full length protein of the Class 1 C. histolyticum collagenase, the DASHES indicate an amino acid deletion, and the DOTS indicate an identical amino acid residue in the first sequence appearing in the first line of the chart. For the chart of FIG. 8B, the NUMBERS, again, refer to an amino acid residue number of the full length protein of the Class 1 C. histolyticum collagenase, the DASHES indicate an amino acid deletion, and the DOTS indicate an identical amino acid residue in the first sequence appearing in the first line of the chart.

These segments show regions of extensive identity and homology to each other indicating a potential conservation of function. Also there is enough homology to the collagen binding domains to hint at an ancestral relationship. Within the amino terminal of the C. histolyticum C1 spacing sequence is the last identified proteolytic sensitive Lys-Ala site (positions 686-687). Within the homologous regions lysine is a very common residue at the first site. In at least one sequence a glutamic acid residue has been observed to replace the alanine that could act as a protective residue to reduce the rate of proteolysis. This segment appears to have a much lower rate of susceptibility to proteolysis then the other sites and its need for modification will need to be evaluated after the more sensitive bonds have been protected.

### Example 8

### Generating Modified Collagenase

The modified collagenase can be generated using methods known in the art of molecular biology and site-directed mutagenesis. A mutagenesis model based on the methods described in U.S. Patent Application Publication US 2003/0162209 A1 {Martin] for quickly incorporating changes is used to modify the Class 1 collagenase gene. In one embodiment, the gene template is a synthetic DNA sequence based on the published protein sequence of Matsushita. See, TABLE 1, above. One pair of PCR primers specific to the cloning vector and at either the N-terminal or C-terminal end of the gene of interest including restriction cleavage sites is generated. Another set of primers containing complementary DNA sequence that contains wild type and mutated bases are also prepared. Two first step PCR reactions are performed followed by one second stage PCR reaction in which a small portion of the two first PCR steps are used as templates to amplify the whole gene of interest including the modified DNA base pairs.

Many suitable expression vectors are known to those skilled in the art. In one specific embodiment, the expression vector contains an antibiotic selectable marker and T7 promoter induction regulatory elements. The vector specific regions on the end of the amplified PCR product are digested with restriction enzymes to cleave the sites introduced during the PCR amplification. In addition, the plasmid vector template is opened by digesting with the same restriction enzymes as the amplified PCR product to linearize the vector. Both vector and mutated gene are agarose gel purified to remove cleaved fragments. The resulting gene and linear vector are ligated using commercially available ligation reagents and procedures. Another approach would be to use commercially available mutagenesis kits.

The ligated constructs containing the mutated collagenase are transformed into BL21-DE3 E. coli cell strain using methods accepted in the field. Resulting colonies are screened for gene insertion and the resulting vector sequenced by standard techniques to confirm sequence and presence of intended modified base pairs. Cell stocks containing the vector with the intended modified C1 collagenase are used to inoculate bacterial culture media. Cell cultures are induced at mid log-phase to express the modified collagenase.

### Example 9

### Purification of Protease Resistant Recombinant C. Histolyticum Collagenase Class 1 Enzyme

The protease resistant enzyme can either be recovered from the cell culture supernatant or from the cells depending upon the choice of vector and cell expression. If the system selected secretes the protein into the media then the cells and debris will be removed by any of a number of techniques (centrifugation, depth filtration or tangential flow filtration as examples). If the desired enzyme is located within the cells then the cells will be recovered from the media. An appropriate lysis technique will liberate the collagenase from the host cell. After removal of the cell debris, the enzyme is ready for purification.

A number of different techniques described in the scientific and patent literature can be used to purify the protease resistant C1 collagenase. These include bulk processes such as concentration, diafiltration along with salt and solvent precipitation. In addition a variety of chromatographic techniques have been used to purify C1 from C. histolyticum collagenase. The techniques of hydrophobic interaction and strong anion exchange chromatographies that are discussed earlier are found to perform well in the purification of the natural enzyme and are expected to work well in this application. If needed a number of other chromatographic methods such as dye ligand affinity, immobilized metal affinity or cation exchange chromatographies can be used. These techniques are amply described in the scientific and patent literature so that anyone skilled in the art of protein purification can reproduce or develop a purification process for this enzyme.

### Example 10

### Uses of Protease Resistant Recombinant C. Histolyticum C1 Collagenase for Wound Debridement, Tissue Remodeling or the Isolation of Cells or Cell Clusters from Tissue or Organs

The uses for protease resistant C. histolyticum collagenase C1 are identical to the natural enzyme. Any protocol which uses the natural C1 enzyme can use the protease resistant form. The only caveat is that because the modified enzyme has enhanced protease stability over the natural enzyme, different (lower) concentrations of the protease resistant enzyme may be required. Anyone skilled in the art of preparing and evaluating enzyme blends will be able to characterize the impact of the improved protease resistant of the C1 enzyme on an application. Listed below are several applications that are presented as examples, and not as an exhaustive list. The compositions are presented as examples and variations of composition are expected to potentially demonstrate improved or deteriorated performance. WOUND DEBRIDEMENT—C. histolyticum protease resistant collagenase C1 and collagenase C2 (natural or recombinant) are prepared in a buffer or solution compatible with live cells and tissue. The two enzymes are blended in a mass ratio of about 1:1. This composition with or without added protease is frozen and lyophilized. The desired mass of this lyophilized powder is mixed with a cream or ointment gel for wound debridement and the acceleration of the healing of decubutus ulcers.

TISSUE REMODELING—C. histolyticum protease resistant collagenase C1 and collagenase C2 (natural or recombinant) are prepared in a buffer or solution compatible with live cells and tissue. The two enzymes are blended in a mass ratio of about 1:1 and diluted to the desired concentration. The blend can then be lyophilized or stored frozen or chilled. TISSUE DISSOCIATION FOR ISOLATING CELLS FROM TISSUE—C. histolyticum protease resistant collagenase C1 and collagenase C2 (natural or recombinant) are prepared in a buffer or solution compatible with live cells and tissue. The C1 and C2 enzymes are to be blended at a ratio experimentally determined for the specific tissue or in a general ratio of about 2 parts C2 to three parts C1. To this is added an enzyme or other material to accelerate the degradation of the non collagen matrix. Depending upon the tissue, a variety of enzymes can be used. This includes but is not limited to general proteases (trypsin, papain, thermolysin or dispase as examples), elastases or hyaluronidases. The composition is then diluted to the desired concentration and placed in contact with the tissue to liberate the desired cells or cell clusters.
TISSUE DISSOCIATION OF HUMAN PANCREAS FOR THE RECOVERY OF ISLETS—C. histolyticum protease resistant collagenase C1 and collagenase C2 (natural or recombinant) are prepared in a buffer or solution compatible with live cells and tissue. The C1 and C2 enzymes are to be blended at a ratio experimentally determined for human pancreas or in a general ratio of about 2 parts C2 to three parts C1. This collagenase blend is then divided into aliquots of about 500 milligrams each. This material is then lyophilized, frozen or retained chilled. Either separately or combined with the collagenase blend is obtained about 12 milligrams of thermolysin. The collagenase and thermolysin blend is then diluted to the desired concentration and used. These techniques for human pancreas dissociation are amply described in the public domain and patent literature so that anyone skilled in the art of islet isolation can use this enzyme composition to recover human islets.
TISSUE DISSOCIATION OF PORCINE PANCREAS FOR THE RECOVERY OF ISLETS—C. histolyticum protease resistant collagenase C1 and collagenase C2 (natural or recombinant) are prepared in a buffer or solution compatible with live cells and tissue. The C1 and C2 enzymes are to be blended at a ratio experimentally determined for porcine pancreas or in a general ratio of about 2 parts C2 to three parts C1. This collagenase blend is then divided into aliquots of about 500 milligrams each. This material is then lyophilized, frozen or retained chilled. Either separately or combined with the collagenase blend is about 30 milligrams of dispase. The collagenase and dispase blend is then diluted to the desired concentration and used. These techniques for porcine pancreas dissociation are amply described in the public domain and patent literature so that anyone skilled in the art of islet isolation can use this enzyme composition to recover porcine islets.

The present invention may be embodied in other specific forms without departing from the spirit or essential attributes thereof and, accordingly, the described embodiments are to be considered in all respects as being illustrative and not restrictive, with the scope of the invention being indicated by the appended claims, rather than the foregoing detailed description, as indicating the scope of the invention as well as all modifications which may fall within a range of equivalency which are also intended to be embraced therein.

1. A native Clostridia histolyticum modified collagenase Class 1 having at least one of amino acid residue selected from the group consisting of lysine (896), lysine (908), leucine (897), alanine (909), lysine (686) and alanine (687) being replaced with an amino acid which provides a proteolytically more stable peptide bond, wherein the selected residue is replaced with an amino acid selected from the group consisting of Gln, Glu, Asp, Asn, Ser, Thr, Gly, Pro and His.

2. The modified collagenase Class 1 according to claim 1, wherein Lys (896) residue is replaced with an amino acid selected from the group consisting of Gln, Glu, Asp, Asn, Ser, Thr, Gly, Pro and His.

3. The modified collagenase Class 1 according to claim 1, wherein Lys (908) residue is replaced with an amino acid selected from the group consisting of Gln, Glu, Asp, Asn, Ser, Thr, Gly, Pro and His.

4. The modified collagenase Class 1 according to claim 1, wherein Lys (686) residue is replaced with an amino acid selected from the group consisting of Gln, Glu, Asp, Asn, Ser, Thr, Gly, Pro and His.

5. The modified collagenase Class 1 according to claim 1, wherein Leu (897) residue is replaced with an amino acid selected from the group consisting of Gln, Glu, Asp, Asn, Ser, Thr, Gly, Pro and His.

6. The modified collagenase Class 1 according to claim 1, wherein Ala (909) residue is replaced with an amino acid selected from the group consisting of Gln, Glu, Asp, Asn, Ser, Thr, Gly, Pro and His.

7. The modified collagenase Class 1 according to claim 1, wherein Ala (687) residue is replaced with an amino acid selected from the group consisting of Gln, Glu, Asp, Asn, Ser, Thr, Gly, Pro and His.

8. The modified collagenase Class 1 according to claim 1, wherein Lys (896) and Lys (908) residues are both replaced with an amino acid selected from the group consisting of Gln, Glu, Asp, Asn, Ser, Thr, Gly, Pro, His and Ala.

9. The modified collagenase Class 1 according to claim 1, wherein Leu (897) and Ala (909) residues are both replaced with an amino acid selected from the group consisting of Gln, Glu, Asp, Asn, Ser, Thr, Gly, Pro and His.

10. The modified collagenase Class 1 according to claim 1, wherein Lys (896), Lys (908), Leu (897) and Ala (909) residues are all replaced with an amino acid selected from the group consisting of Gln, Glu, Asp, Asn, Ser, Thr, Gly, Pro and His.

11. A native Clostridia histolyticum modified collagenase Class 1 wherein at least one of the residues selected form the group consisting of lysine (896), lysine (908), leucine (897), alanine (909), lysine (686) and alanine (687) has been deleted from the protein.

12. The modified collagenase Class 1 of claim 11 wherein, Lys (896) has been deleted from the protein.

13. The modified collagenase Class 1 of claim 11, wherein Lys (908) has been deleted from the protein.

14. The modified collagenase Class 1 of claim 11, wherein Leu (897) has been deleted from the protein.

15. The modified collagenase Class 1 of claim 11, wherein Ala (909) has been deleted from the protein.

16. The modified collagenase Class 1 of claim 11, wherein Lys (896) and Lys (908) have been deleted from the protein.

17. The modified collagenase Class 1 of claim 11, wherein Leu (897) and Ala (909) have been deleted from the protein.

18. The modified collagenase Class 1 of claim 11, wherein Lys (896), Lys (908), Leu (897) and Ala (909) have been deleted from the protein.

19. A native collagenase Class 1 from Clostridia and Bacillus species which contain homologous protease sensitive residues wherein at least one of the homologous protease sensitive residues have been replaced with an amino acid which provides a proteolytically more stable peptide bond, wherein the residue is replaced with an amino acid selected from the group consisting of Gln, Glu, Asp, Asn, Ser, Thr, Gly, Pro, His and Ala.

20. A method of using the modified collagenase Class 1 for the dissociation of tissue for the recovery of viable primary cells or cell clusters, wound debridement and tissue remodeling or regeneration.

21. A method of using the modified collagenase Class 1 along with modified or unmodified collagenase Class 2 and other proteolytic enzymes for the dissociation of tissue for the recovery of viable primary cells or cell clusters, wound debridement and tissue remodeling or regeneration.

22. A method of using the modified collagenase Class 1 along with modified or unmodified collagenase Class 2 and other proteolytic enzymes for the dissociation of pancreatic tissue for the recovery of functional islets.

23. A method of using the modified collagenase Class 1 along with modified or unmodified collagenase Class 2 and thermolysin for the dissociation of human pancreatic tissue for the recovery of functional human islets.

24. A method of using the modified collagenase Class 1 along with modified or unmodified collagenase Class 2 and dispase for the dissociation of porcine pancreatic tissue for the recovery of functional porcine islets.

25. A recombinant DNA molecule comprising a DNA sequence encoding a native collagenase Class 1 molecule consisting of a catalytic domain attached to at least one linking domain which is attached to at least two collagen binding domains all of which are homologous to the corresponding domains in C. histolyticum collagenase Class 1 wherein at least one of the protease sensitive bonds identified has been modified to provide a proteolytically more stable peptide bond.

26. A cell containing the modified recombinant DNA molecule of claim 25.

27. A method for the production of native Clostridia histolyticum modified collagenase Class 1 comprising the steps of
(a) transforming a cell with recombinant DNA molecule having a DNA sequence encoding a native collagenase class 1 molecule consisting of a catalytic domain attached to at least one linking domain which is attached to at least two collagen binding domains all of which are homologous to the corresponding domains in C. histolyticum collagenase class 1 wherein at least one of the protease sensitive bonds identified has been modified to provide a proteolytically more stable peptide bond;
(b) culturing the transformed cells of step (a); and,
(c) isolating the native modified Clostridia histolyticum collagenase Class 1, expressed in the cultured transformed cells of step (b).
